(19) United States
(12) Patent Application Publication
(10) Pub. No.: US 2026/0013927 A1
(43) Pub. Date: Jan. 15, 2026

(54) APPLYING ENERGY TO AN ELECTROSURGICAL INSTRUMENT

(71) Applicant: CMR Surgical Limited, Cambridge (GB)

(72) Inventors: David William Haydn Webster-Smith, Cambridge (GB); Dominic Martin McBrien, Cambridge (GB); Jonathan Peter Waite, Cambridge (GB)

(73) Assignee: CMR Surgical Limited, Cambridge, OT (GB)

(21) Appl. No.: 18/881,086

(22) PCT Filed: Jun. 23, 2023
(86) PCT No.: PCT/GB2023/051659

§ 371 Date: Jan. 3, 2025

(30) Foreign Application Priority Data
Jul. 5, 2022 (GB) 2209901.4

(51) Int. Cl. A61B 18/12; A61B 18/00; A61B 34/37

(52) U.S. Cl. CPC A61B 18/1206; A61B 34/37; A61B 2018/00601; A61B 2018/00648; A61B 2018/00672; A61B 2018/00678; A61B 2018/00702; A61B 2018/0072; A61B 2018/00761; A61B 2018/00767; A61B 2018/00827; A61B 2018/00869; A61B 2018/00875; A61B 2018/00892; A61B 2018/00982

(57) ABSTRACT

A control system for controlling the application of energy from an electrosurgical generator to a robotic electrosurgical instrument during a cut mode in which the electrosurgical instrument is used to cut tissue. The control system: controls the electrosurgical generator to apply energy to the electrosurgical instrument during a first cut phase; detects a trigger condition has been met during the first cut phase; upon detecting that the trigger condition has been met, starts a timer; and upon a predetermined time period elapsing after the timer has started, controls the electrosurgical generator to switch from the first cut phase to a second cut phase, the second cut phase differing from the first cut phase by one or more energy parameters.

### BACKGROUND

It is known to use robots for assisting and performing surgery. FIG. 1 illustrates a typical surgical robotic system. A surgical robot 100 consists of a base 102, an arm 104 and an instrument 106. The base supports the robot, and may itself be attached rigidly to, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation. The end effector engages in a surgical procedure.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives inputs from the surgeon input device(s) 114 and converts these to control signals to move the joints of the robot arm 104 and instrument 106. The control system sends these control signals to the robot, where the corresponding joints are driven accordingly.

A variety of end effectors are known, each adapted to perform a particular surgical function. An electrosurgical instrument is a surgical instrument adapted to perform electrosurgery. Electrosurgery is the passing of a high frequency, for example radio frequency, current through tissue to cause a desired effect. Examples of that desired effect are cutting the tissue and coagulating the tissue.

There are two types of electrosurgery: monopolar and bipolar. In monopolar electrosurgery the high frequency current passes through the patient from an active electrode of the monopolar electrosurgical instrument to a separate return electrode placed on the patient. In bipolar electrosurgery the active and return electrodes are both within the bipolar electrosurgical instrument. The current passes through the patient from the active electrode of the bipolar electrosurgical instrument to the return electrode of the bipolar electrosurgical instrument.

Electrosurgical instruments receive the high frequency current from an electrosurgical generator Electrosurgical generators are generally capable of generating multiple different current waveforms to achieve different surgical effects. For example, electrosurgical generators can be configured to generate COAG and CUT waveforms. The COAG waveform consists of bursts of radio frequency, which when used at a low power setting causes a desiccation effect, and when used at a high-power setting causes a fulguration effect. The CUT waveform is a continuous waveform at higher voltage than COAG, which causes the tissue to be cut.

Increasing the voltage of the CUT waveform applied by an electrosurgical generator to a bipolar electrosurgical instrument increases the performance of the cutting action, however it can cause sparking to occur. The sparking in turn can cause tissue surrounding the bipolar electrosurgical instrument to burn.

There is therefore a need for a mechanism for safely improving the performance of the cutting action of a bipolar electrosurgical instrument without causing unwanted sparking.

### SUMMARY OF THE INVENTION

According to an aspect of the invention, there is provided a control method for controlling the application of energy from an electrosurgical generator to a robotic electrosurgical instrument during a cut mode in which the electrosurgical instrument is used to cut tissue, the control method comprising: controlling the electrosurgical generator to apply energy to the electrosurgical instrument during a first cut phase; detecting a trigger condition has been met during the first cut phase; upon detecting that the trigger condition has been met, starting a timer; and upon a predetermined time period elapsing after the timer has started, controlling the electrosurgical generator to switch from the first cut phase to a second cut phase, the second cut phase differing from the first cut phase by one or more energy parameters.

The trigger condition may be one or more parameter threshold having been reached or passed.

The trigger condition may comprise the voltage applied by the electrosurgical generator to the electrosurgical instrument reaching a voltage threshold.

The trigger condition may comprise both: the current applied by the electrosurgical generator to the electrosurgical instrument reaching or dropping below a current threshold; and the phase angle between the current and voltage applied by the electrosurgical generator to the electrosurgical instrument reaching or exceeding a phase angle threshold.

The trigger condition may comprise the tissue impedance reaching or exceeding a tissue impedance threshold.

The first cut phase may differ from the second cut phase by one or more of the following energy parameters: voltage limit of the voltage applied by the electrosurgical generator, power limit of the power applied by the electrosurgical generator, and current limit of the current applied by the electrosurgical generator.

The first cut phase may be the initial application of energy from zero voltage to a first voltage, and the second cut phase may be the application of energy at a second voltage.

The first voltage may be a peak voltage of the cut mode, and the second voltage may be lower than the first voltage.

The second voltage may be a peak voltage of the cut mode, and the first voltage may be lower than the second voltage.

The trigger condition may comprise the voltage applied by the electrosurgical generator to the electrosurgical instrument reaching or exceeding a voltage threshold; and once the voltage threshold is reached or exceeded, the control method may comprise applying the first voltage until the predetermined time period has elapsed.

The control method may further comprise: detecting a further trigger condition has been met during the second cut phase; upon detecting that the further trigger condition has been met, starting a further timer; and upon a further predetermined time period elapsing after the further timer has started, controlling the electrosurgical generator to switch from the second cut phase to a third cut phase, the third cut phase differing from the second cut phase by one or more energy parameters.

The third cut phase may be the application of zero voltage.

The further trigger condition may comprise both: the current applied by the electrosurgical generator to the electrosurgical instrument reaching or dropping below a current threshold; and the phase angle between the current and voltage applied by the electrosurgical generator to the electrosurgical instrument reaching or exceeding a phase angle threshold.

The first cut phase may be the application of energy at a first voltage, and the second cut phase may be the application of zero voltage.

The first voltage may be a peak voltage of the cut mode.

The first voltage may be lower than a peak voltage of the cut mode.

The trigger condition may comprise both: the current applied by the electrosurgical generator to the electrosurgical instrument reaching or dropping below a current threshold; and the phase angle between the current and voltage applied by the electrosurgical generator to the electrosurgical instrument reaching or exceeding a phase angle threshold; and once the trigger condition is met, the control method may comprise applying the first voltage until the predetermined time period has elapsed.

The control method may further comprise: controlling the electrosurgical generator to apply energy to the electrosurgical instrument during a third cut phase; detecting a further trigger condition has been met during the third cut phase; upon detecting that the further trigger condition has been met, starting a further timer; and upon a further predetermined time period elapsing after the further timer has started, controlling the electrosurgical generator to switch from the third cut phase to the first cut phase, the third cut phase differing from the first cut phase by one or more energy parameters.

The third cut phase may be the initial application of energy from zero voltage to a third voltage.

The third voltage may be a peak voltage of the cut mode.

The further trigger condition may comprise the voltage applied by the electrosurgical generator to the electrosurgical instrument reaching or exceeding a voltage threshold.

The further predetermined time period may be shorter than the predetermined time period.

The further predetermined time period may be longer than the predetermined time period.

The further predetermined time period may be the same as the predetermined time period.

The control method may further comprise receiving measurements from sensors of the electrosurgical generator, the measurements comprising voltage measurements and/or current measurements.

The control method may further comprise outputting a cut complete indication, that cut complete indication being different to the video feed from a camera recording the tissue being cut.

The control method may comprise controlling the electrosurgical generator to apply energy continuously in each cut phase.

The robotic electrosurgical instrument may be a bipolar electrosurgical instrument.

### BRIEF DESCRIPTION OF THE FIGURES

The present invention will now be described by way of example with reference to the accompanying drawings. In the drawings:

FIG. 1 illustrates a surgical robot system for performing a surgical procedure;

FIG. 2 illustrates a surgical robot;

FIG. 3 illustrates an electrosurgical instrument;

FIG. 4 illustrates the distal end of a bipolar electrosurgical instrument;

FIG. 5 illustrates a surgical robot system for performing an electrosurgical procedure;

FIG. 6 illustrates a graph of a known CUT operation;

FIG. 7 is a flowchart illustrating a method of controlling an electrosurgical generator to apply energy to a robotic electrosurgical instrument during a CUT mode; and

FIG. 8 is a graph illustrating phases of a CUT operation.

### DETAILED DESCRIPTION

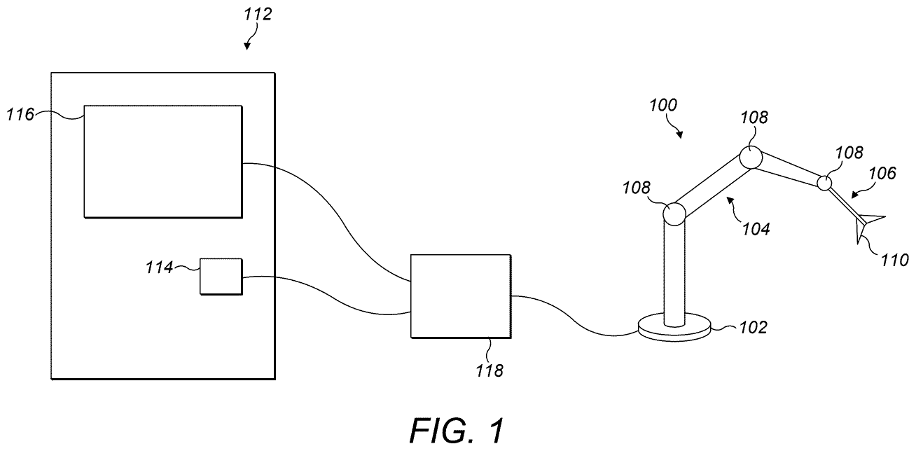

The following describes a method of controlling the application of energy from an electrosurgical generator to a robotic electrosurgical instrument. In particular, the application of energy during a cut mode of the robot is discussed. The robotic electrosurgical instrument is mounted to a surgical robotic arm. The surgical robotic arm and electrosurgical instrument together form part of a surgical robotic system of the type illustrated in FIG. 1.

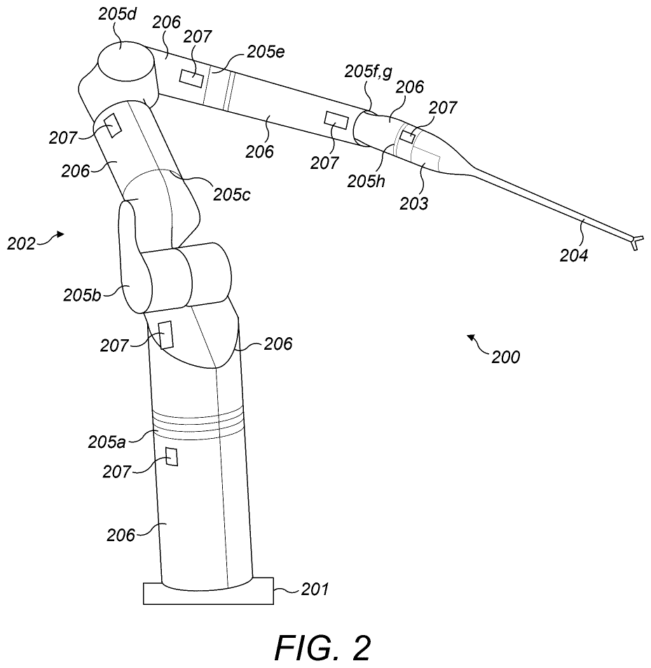

FIG. 2 illustrates an example robot 200. The robot comprises a base 201 which is fixed in place when a surgical procedure is being performed. Suitably, the base 201 is mounted to a chassis. That chassis may be a cart, for example a bedside cart for mounting the robot at bed height. Alternatively, the chassis may be a ceiling mounted device, or a bed mounted device.

A robot arm 202 extends from the base 201 of the robot to a terminal link 203 to which a surgical instrument 204 can be attached. The arm is flexible. It is articulated by means of multiple flexible joints 205 along its length. In between the joints are rigid arm links 206. The arm in FIG. 2 has eight joints. The joints include one or more roll joints (which have an axis of rotation along the longitudinal direction of the arm members on either side of the joint), one or more pitch joints (which have an axis of rotation transverse to the longitudinal direction of the preceding arm member), and one or more yaw joints (which also have an axis of rotation transverse to the longitudinal direction of the preceding arm member and also transverse to the rotation axis of a co-located pitch joint). In the example of FIG. 2: joints 205a, 205c, 205e and 205h are roll joints; joints 205b, 205d and 205f are pitch joints; and joint 205g is a yaw joint. Pitch joint 205f and yaw joint 205g have intersecting axes of rotation. The order of the joints from the base 201 to the terminal link 203 of the robot arm is thus: roll, pitch, roll, pitch, roll, pitch, yaw, roll. However, the arm could be jointed differently. For example, the arm may have fewer than eight or more than eight joints. The arm may include joints that permit motion other than rotation between respective sides of the joint, for example a telescopic joint. The robot comprises a set of drivers 207. Each driver 207 has a motor which drives one or more of the joints 205. The terminal link 203 of the robot arm comprises a drive assembly for interfacing and driving a surgical instrument. The drive assembly comprises drive assembly interface elements which engage with corresponding instrument interface elements of an instrument interface of the surgical instrument. The drive assembly interface elements are driven by drivers 207. As the drive assembly interface elements move they move the instrument interface elements they are engaged with, thereby transferring drive from the drive assembly of the robot arm to the instrument interface of the instrument.

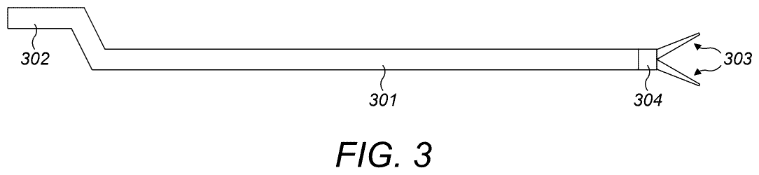

FIG. 3 illustrates a bipolar electrosurgical instrument 300. The bipolar electrosurgical instrument has an elongate profile, with a shaft 301 spanning between its proximal end which is attached to the robot arm and its distal end which accesses the surgical site within the patient body. Suitably, the shaft is rigid. The shaft may be straight. The proximal end of the surgical instrument and the instrument shaft may be rigid with respect to each other and rigid with respect to the distal end of the robot arm when attached to it. At the proximal end of the instrument, the shaft 301 is connected to an instrument interface 302. The instrument interface engages with the drive assembly interface at the distal end of the robot arm. Drive is transferred from the robot arm to the surgical instrument at this interface. At the distal end of the surgical instrument, the distal end of the shaft is connected to an electrosurgical end effector 303 by an articulation 304. The articulation 304 permits the end effector 303 to move relative to the shaft 301. Suitably, the articulation permits the end effector 303 to move with at least two degrees of freedom relative to the shaft 301.

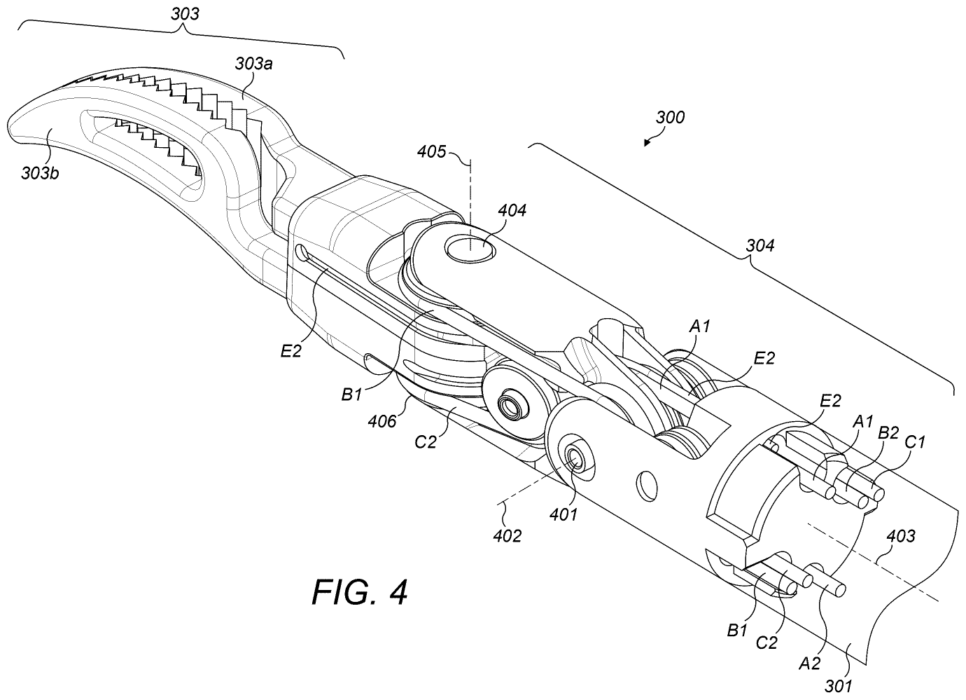

FIG. 4 illustrates the distal end of the bipolar electrosurgical instrument 300 of FIG. 3 in more detail. The instrument has two end effector elements 303a, 303b which are depicted as opposing jaws. It will be understood that this is for illustrative purposes only. The electrosurgical end effector elements may take any suitable shape. As another example, the electrosurgical end effector elements may be scissors. Articulation 304 comprises joints which permit the electrosurgical end effector 303 to move relative to the shaft 301. A first joint 401 permits the electrosurgical end effector 303 to rotate about a first axis 402. The first axis 402 is transverse to the longitudinal axis of the shaft 403. A second joint 404 permits the first end effector element 303a to rotate about a second axis 405. The second axis 405 is transverse to the first axis 402. A third joint 406 permits the second end effector element 303b to rotate about the second axis 405. Suitably, rotation of the first end effector element 303a about the second axis 405 is independent of rotation of the second end effector element 303b about the second axis 405. Articulation of the first, second and third joints enables the electrosurgical end effector to take a range of attitudes relative to the shaft. The joints of the articulation are driven by driving elements. The first joint 401 is driven by a first pair of driving elements A1,A2. The second joint 404 is driven by a second pair of driving elements B1,B2. The third joint 406 is driven by a third pair of driving elements C1,C2. Those driving elements are in turn driven by the instrument interface elements of the instrument interface.

The electrosurgical end effector 303 is powered by a pair of electrosurgical elements E1,E2. The electrosurgical elements E1,E2 extend from the instrument interface, through the shaft to the electrosurgical end effector 303. The electrosurgical elements connect to separate parts of the electrosurgical end effector 303 which are insulated from each other. In the example of FIG. 4, the first electrosurgical element E1 is connected to the first electrosurgical end effector element 303a, and the second electrosurgical element E2 is connected to the second electrosurgical end effector element 303b. Suitably, the first electrosurgical element E1 terminates at the first electrosurgical end effector element 303a, and the second electrosurgical element E2 terminates at the second electrosurgical end effector element 303b. The first electrosurgical element E1 may be overmoulded with insulation material where it terminates at the first electrosurgical end effector element. The second electrosurgical element E2 may be overmoulded with insulation material where it terminates at the second electrosurgical end effector element. When the two electrosurgical end effector elements are closed onto tissue, current can be applied via the electrosurgical elements to cut or coagulate the tissue captured between the end effector elements. The electrosurgical elements E1,E2 are connected to an electrosurgical power cable at the instrument interface. That electrosurgical power cable is connected at its other end to an electrosurgical generator. The electrosurgical generator provides energy to the electrosurgical elements E1,E2 to operate the electrosurgical instrument.

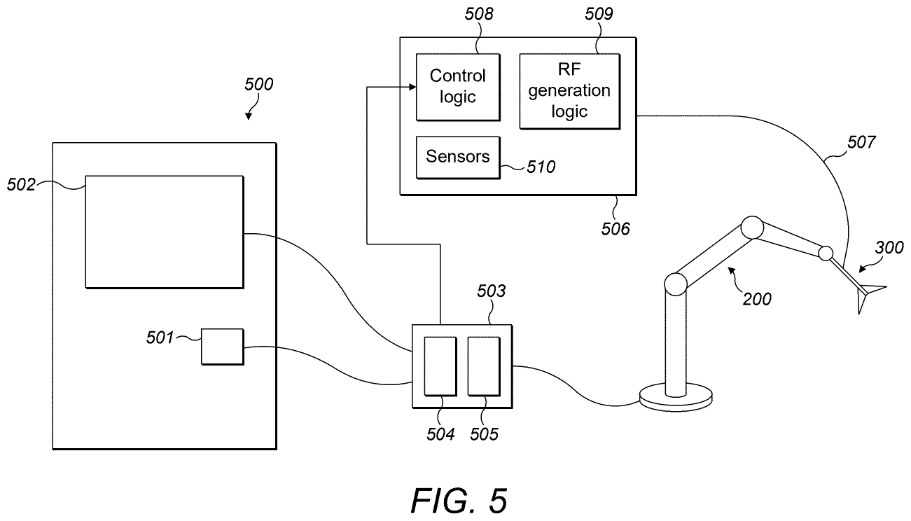

FIG. 5 illustrates a surgical robotic system for implementing an electrosurgical procedure. The robot arm 200 may be as described above with respect to FIG. 2, and the electrosurgical instrument 300 as described above with respect to FIGS. 3 and 4. A surgeon controls the surgical robot 200 via a remote surgeon console 500. The surgeon console 500 comprises one or more surgeon input devices 501. These may take the form of a hand controller, other inputs for activation by fingers or hands, foot pedal, voice activated input, gesture input, eye movement input etc. The surgeon console also comprises a display 502. A control system 503 connects the surgeon console 500 to the surgical robot 200. The control system 503 comprises a processor 504 and memory 505. Memory 505 stores in a non-transient way software that is executable by the processor 504 to control the operation of the motors 207 to cause the robot arm 200 and instrument 300 to operate in the manner described herein. The control system receives inputs from the surgeon input device(s) 501 and converts these to control signals to move the joints of the robot arm and electrosurgical instrument. The control system sends these control signals to the robot, where the corresponding joints and/or drive assembly interface elements are driven accordingly.

The surgical robot system further comprises an electrosurgical generator 506 which is connected to the electrosurgical elements E1,E2 in the instrument interface by an electrosurgical power cable 507. Alternatively, the electrosurgical generator 506 may be connected to an electrosurgical connection unit at the terminal end of the robot arm, and the electrosurgical connection unit connected to the electrosurgical elements E1,E2 in the instrument interface via an electrical cable.

The electrosurgical generator 506 generates electrosurgical signals for driving the electrosurgical instrument 300. The electrosurgical generator may generate different current waveforms. For example, the electrosurgical generator may be configured to generate a COAG waveform which consists of bursts of radio frequency, which when used at a low power setting causes a desiccation effect, and when used at a high-power setting causes a fulguration effect. The electrosurgical generator may be configured to generate a typical CUT waveform which consists of a continuous waveform at a higher voltage than COAG, which causes the tissue to be cut. The electrosurgical generator 506 comprises any suitable means for configuring the waveforms to be generated. For example, an electrosurgical generator 506 may comprise a user interface that comprises, for example, switches, buttons, dials etc., which enable a user to configure each supported waveform. Alternatively, the electrosurgical generator 506 may be configured electronically, such as via a control signal transmitted to the electrosurgical generator from a computing device. For example, the electrosurgical generator 506 may be connected to the control system 503 and the waveforms commanded by the surgeon input device(s) 501.

The electrosurgical generator 506 comprises control logic 508 which is configured to receive activation signals indicating which waveform is to be generated by the electrosurgical generator 506. In response to the control logic 508 receiving an activation signal indicating a waveform, RF generation logic 509 generates the waveform. This waveform is then output from the electrosurgical generator 506 as the electrosurgical signal to the electrosurgical instrument via the power cable 507. When an activation signal is detected by the control logic 508, in addition to causing an electrosurgical signal with the desired waveform to be output, the control logic 508 may cause a feedback signal to be output to alert the user of the activation of a particular waveform. The feedback may be in the form of visual feedback (e.g. an indicator light on a display panel of the electrosurgical generator 506) or audible feedback (e.g. a tone).

The electrosurgical generator 506 may receive activation signals from the control system 503. The control system 503 may generate the activation signals in response to the surgeon providing input via the surgeon input device(s) 501. These activation signals may be sent from the control system 503 directly to the electrosurgical generator 506 either by a wired or wireless connection. Alternatively, in the example in which an electrosurgical connection unit is connected to the robot arm, the control system 503 may send the activation signals to the electrosurgical connection unit, and the electrosurgical connection unit transmit these activation signals to the electrosurgical generator 506 either by a wired or wireless connection.

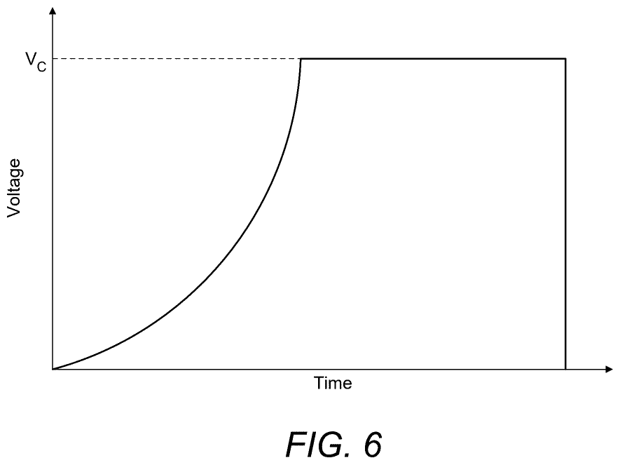

FIG. 6 illustrates a typical CUT waveform generated by an electrosurgical generator. The voltage of the CUT waveform is plotted against time on the x-axis. The CUT waveform is a continuous waveform which has a higher voltage than a typical COAG waveform. The CUT waveform is typically a constant RMS voltage/current waveform once the desired voltage/current has been reached. This is shown on FIG. 6 as the constant RMS voltage V_{c}. Once the cut has finished, the surgeon commands the end of the CUT operation, which causes the CUT waveform to cease being applied to the electrosurgical instrument.

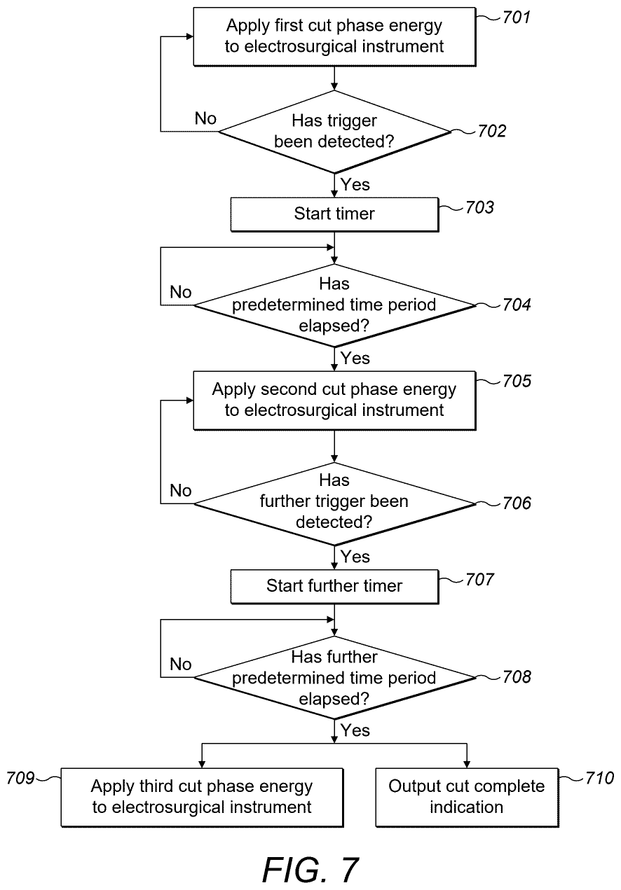

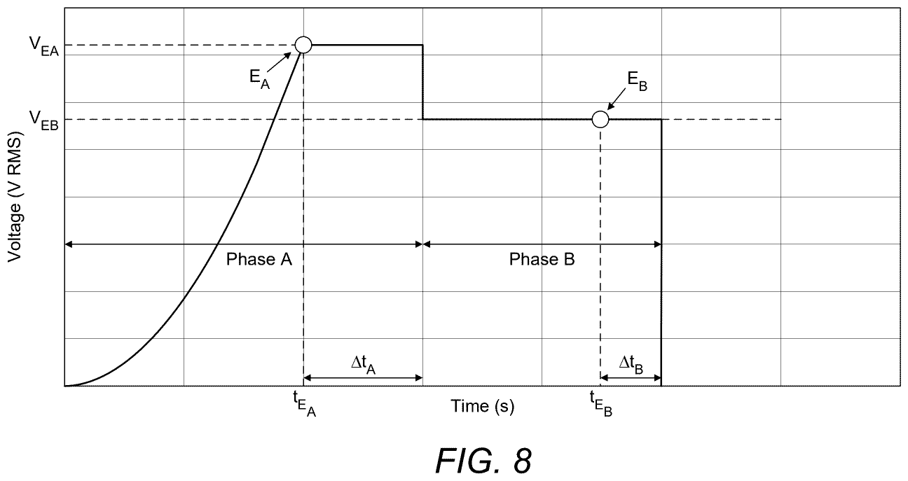

FIG. 7 is a flowchart illustrating a method of controlling an electrosurgical generator to apply energy to a robotic electrosurgical instrument during a CUT mode to cut tissue. This method may be implemented for a bipolar robotic electrosurgical instrument. The CUT waveform comprises at least two distinct phases of continuous energy application. These two cut phases differ by one or more of the following energy parameters: voltage limit of the voltage applied by the electrosurgical generator, power limit of the power applied by the electrosurgical generator, current limit of the current applied by the electrosurgical generator, duty cycle of the energy applied by the electrosurgical generator, pulse length of the energy applied by the electrosurgical generator, and frequency of the electrosurgical energy signal applied by the electrosurgical generator. FIG. 8 is a graph illustrating an exemplary CUT waveform generated by the electrosurgical generator. In the example of FIG. 8, the two phases of the CUT waveform are labelled Phase A and Phase B.

Initially, the electrosurgical generator 506 receives an activation signal for a CUT waveform. This activation signal may be received from the control system 503 in response to an input from the surgeon input device 501, either directly or via an electrosurgical connection unit on the robot arm. Alternatively, the activation signal may be received as a direct input on the electrosurgical generator 506. Alternatively, the activation signal may be received from an electrosurgical connection unit on the robot arm in response to an input on the electrosurgical connection unit or robot arm. The control logic 508 of the electrosurgical generator 506 responds to the activation signal at step 701 by controlling the RF generation logic 509 to generate a first cut phase signal and output this to the electrosurgical instrument. FIG. 8 illustrates an exemplary CUT waveform. Initially, during the first cut phase, referred to on FIG. 8 as Phase A, the voltage ramps up from zero. During this first cut phase, the electrosurgical generator may operate with maximum voltage VA, power PA and current limits IA.

The voltage and current of the electrosurgical signal applied by the electrosurgical generator to the electrosurgical instrument are measured by sensors. Suitably, these voltage and current sensors 510 are located in the electrosurgical generator to directly measure the voltage and current of the waveform generated by the circuitry of the electrosurgical generator. Alternatively, or in addition, voltage and current sensors may be located at the electrosurgical instrument. In this case, the voltage and current sensors measure the voltage and current of the electrosurgical signal received by the electrosurgical instrument from the electrosurgical generator. In the case that an electrosurgical connection unit is used on the robot arm, the electrosurgical connection unit may receive the electrosurgical signal from the electrosurgical generator and route this to the electrosurgical instrument. In this case, the voltage and current sensors may be located at any one, or combination, of the electrosurgical generator, the electrosurgical connection unit, and the electrosurgical instrument.

Whilst applying the CUT waveform to the electrosurgical instrument during the first cut phase, an assessment is made as to whether a trigger has been detected. This is step 702 on FIG. 7. The trigger condition may be, for example, that a parameter threshold has been reached or passed. In the example of FIG. 8, the trigger condition is that the voltage V of the CUT waveform in phase A has reached or exceeded a voltage threshold, V_{EA}. V≥V_{EA}. V_{EA} may be in the range 0-1000V. V_{EA} may be in the range 0-500V. V_{EA} may be 450V. This happens a time t_{EA} after the beginning of the first cut phase, phase A. The trigger condition is monitored by a control device: either control logic 508 of the electrosurgical generator or control system 503 or the electrosurgical connection unit. The control device that is monitoring whether the trigger condition has been met receives the voltage and current measurements from the sensors described above. In the case that the trigger condition is a voltage/current threshold having been reached/exceeded/dropped below, the control device compares the voltage/current measurements it receives to the voltage/current threshold, and determines whether the trigger condition has been met using the result of the comparison. The control device may perform a more complicated calculation in order to determine whether the trigger condition has been met. For example, if the trigger condition is an impedance threshold, then the control device may first calculate an impedance value from the voltage/current measurements, and then compare that calculated impedance value to the impedance threshold, and determine whether the trigger condition has been met using the result of the comparison.

If, at step 702, the trigger condition has not been met, then the control method returns to step 701. Note that the energy applied by the electrosurgical generator to the electrosurgical instrument in step 701 continues whilst the trigger condition is being assessed in step 702. If, at step 702, the trigger condition has been met, then the control device starts a timer at step 703. This timer is started as soon as the trigger condition is determined to have been met. In the example of FIG. 8, this timer is started at t_{EA}. Whilst the timer is activated, the control device continues to control the electrosurgical generator to generate and apply energy to the electrosurgical instrument according to the first cut phase. Suitably, during the period of the first cut phase whilst the timer is activated, the electrosurgical generator generates a constant RMS voltage electrosurgical signal. This is illustrated in the example of FIG. 8. The voltage of the CUT waveform in FIG. 8 ramps up from zero to the voltage V_{EA} during the first cut phase. The voltage V_{EA} is the threshold voltage trigger condition, which when met causes the control device to respond by starting the timer. The voltage V_{EA} is also the constant RMS voltage that is maintained during the remainder of the first cut phase. In the example of FIG. 8, the voltage V_{EA} is the peak voltage of the CUT waveform.

At step 704, the control device determines whether a predetermined time period has elapsed since the timer started, as measured by the timer. In FIG. 8, the predetermined time period is shown as Δt_{A}. The predetermined time period may be a set period of time. For example, the predetermined time period may be a value between 0 and 5 seconds. The predetermined time period may be selected by the user. Alternatively, the predetermined time period may be a function of another parameter. For example:
1. The predetermined time period may be a function of the type of tissue being cut. For example, thicker or stiffer tissues may be assigned a longer predetermined time period than thinner or softer tissues.
2. The predetermined time period may be a function of the time taken for the trigger condition in the first cut phase to be met. In other words, the time between the beginning of the electrosurgical signal activation and the trigger condition of the first cut phase being met.
3. The predetermined time period may be a function of the maximum voltage for the first cut phase.
4. The predetermined time period may be a function of the tissue impedance when the trigger condition of the first cut phase is met.
5. The predetermined time period may be a function of a time period of the second cut phase of the electrosurgical signal.

Upon the predetermined time period elapsing, the control device controls the electrosurgical generator to switch from the first cut phase to a second cut phase at step 705. In the example of FIG. 8, the control device controls the electrosurgical generator to switch from the first cut phase, phase A, to the second cut phase, phase B, as soon as the time t satisfies the following inequality: t≥t_{EA}+Δt_{A}. The second cut phase differs from the first cut phase by one or more energy parameters. During this second cut phase, the electrosurgical generator may operate with maximum voltage V_{B}, power P_{B} and current limits I_{B}. These limits may be different to the corresponding limits of the first cut phase. Each of the second cut phase limits individually may be higher, lower or the same as the corresponding limit of the first cut phase.

In the second cut phase, the control logic 508 of the electrosurgical generator 506 controls the RF generation logic 509 to generate a second cut phase signal and output this to the electrosurgical instrument. The second cut phase signal may be a constant RMS voltage signal. In the exemplary CUT waveform of FIG. 8, during the second cut phase, phase B, the voltage is constant. In FIG. 8, the constant RMS voltage V_{EB} of phase B is lower than the constant RMS voltage V_{EA} of phase A. V_{EB} may be in the range 0-1000V. V_{EB} may be in the range 0-500V. V_{EB} may be 280V. Thus, at the time t=t_{EA}+Δt_{A}, the voltage of the CUT waveform steps down from V_{EA} to V_{EB}. In an alternative example, the constant voltage of the second cut phase may be higher than the constant voltage of the first cut phase. In this example, the constant voltage of the second cut phase may be the peak voltage of the CUT waveform.

Whilst applying the CUT waveform to the electrosurgical instrument during the second cut phase, an assessment is made as to whether a further trigger has been detected. This is step 706 on FIG. 7. The further trigger condition may be, for example, that a parameter threshold has been reached or passed. In the example of FIG. 8, the further trigger condition comprises both: (i) that the current I of the CUT waveform in phase B has reached or dropped below a current threshold, I_{EB}, I≤I_{EB}, and (ii) that the phase angle ϕ between the current and voltage has reached or exceeded a phase angle threshold, ϕ_{EB}, ϕ≥ϕ_{EB}. The current threshold may be a very low current. For example, I_{EB}=0. The phase angle threshold may be high. For example, I_{EB}=90°. Alternatively, the further trigger condition may be that a tissue impedance threshold has been reached or exceeded. In the example of FIG. 8, the further trigger condition is met at a time t=t_{EB} after the beginning of the first cut phase, phase A.

The further trigger condition of the second cut phase is monitored and assessed by a control device as described above with respect to the trigger condition of the first cut phase. In the case of the phase angle threshold of FIG. 8, the control device calculates a phase angle value from the difference in phase between voltage and current measurements taken at the same time, and then compares that calculated phase angle to the phase angle threshold. For the example of FIG. 8, the control device only determines the trigger condition to be met if both the measured current I satisfies the inequality I≤I_{EB} and the calculated phase angle value satisfies the inequality ϕ≥ϕ_{EB}.

If, at step 706, the further trigger condition has not been met, then the control method returns to step 705. Note that the energy applied by the electrosurgical generator to the electrosurgical instrument in step 705 continues whilst the further trigger condition is being assessed in step 706. If, at step 706, the further trigger condition has been met, then the control device starts a further timer at step 707. This further timer is started as soon as the further trigger condition is determined to have been met. In the example of FIG. 8, this further timer is started at t=t_{EB}. Whilst the further timer is activated, the control device continues to control the electrosurgical generator to generate and apply energy to the electrosurgical instrument according to the second cut phase. Suitably, during the period of the second cut phase whilst the further timer is activated, the electrosurgical generator generates a constant RMS voltage electrosurgical signal. This is illustrated in the example of FIG. 8. The voltage of the CUT waveform in FIG. 8 during the second cut phase is V_{EB}. This voltage V_{EB} is maintained throughout the second cut phase.

At step 708, the control device determines whether a further predetermined time period has elapsed since the further timer started, as measured by the further timer. In FIG. 8, the further predetermined time period is shown as Δt_{B}. The further predetermined time period may be a set period of time. For example, the further predetermined time period may be a value between 0 and 5 seconds. The predetermined time period may be selected by the user. Alternatively, the further predetermined time period may be a function of another parameter. For example:
1. The further predetermined time period may be a function of the type of tissue being cut. For example, thicker or stiffer tissues may be allocated a longer further predetermined time period than thinner or softer tissues.
2. The predetermined time period may be a function of the time taken for the trigger condition in the second cut phase to be met. In other words, the time between the beginning of the second cut phase and the trigger condition of the second cut phase being met.
3. The predetermined time period may be a function of the maximum voltage for the second cut phase.
4. The predetermined time period may be a function of the tissue impedance when the trigger condition of the second cut phase is met.
5. The predetermined time period may be a function of a time period of the first cut phase of the electrosurgical signal.

Upon the further predetermined time period elapsing, the control device controls the electrosurgical generator to switch from the second cut phase to a third cut phase at step 709. In the example of FIG. 8, the control device controls the electrosurgical generator to switch from the second cut phase, phase B, to the third cut phase, as soon as the time t satisfies the following inequality: t≥t_{EB}+Δt_{B}. The third cut phase differs from the second cut phase by one or more energy parameters. During this third cut phase, the electrosurgical generator may apply zero volts. In other words, the third cut phase may be an end of the cut operation.

In the third cut phase, the control logic 508 of the electrosurgical generator 506 controls the RF generation logic 509 to generate a third cut phase signal and output this to the electrosurgical instrument. In the case that the third cut phase is the end of the cut operation, then the control logic 508 controls the RF generation logic 509 to step the voltage of the CUT waveform down from the voltage of the second cut phase, for example V_{EB} in FIG. 8, to zero volts at t=t_{EB}+Δt_{B}.

At, or immediately following, the switch from the second cut phase to the third cut phase, a cut complete indication may be output. This may be output from one or more of: the electrosurgical generator 506, the control system 503, the surgeon console 500 and the electrosurgical connection unit. The cut complete indication is different to the video feed from the endoscopic camera filming the operation. The cut complete indication may be an audible signal, such as a tone. The cut complete indication may be a visual signal, such as an icon on the surgeon's display 502. The cut complete indication may be a haptic signal, such as a vibration of the surgeon's hand controller.

The predetermined time period of the first cut phase and the further predetermined time period of the second cut phase may be the same length of time. Alternatively, the predetermined time period of the first cut phase may be shorter than the further predetermined time period of the second cut phase. Alternatively, the predetermined time period of the first cut phase may be longer than the further predetermined time period of the second cut phase.

In the CUT mode operation described above, the electrosurgical generator continuously supplies energy to the electrosurgical instrument during the described cut phases. This is following initiation of the cutting operation by the surgeon by means of an activation input on the surgeon input device 501. If the surgeon commands the cutting operation to stop whilst in any of the cut phases described herein, for example by manipulation of an input on the surgeon input device 501, then the control system 503 controls the electrosurgical generator to stop outputting the CUT waveform to the electrosurgical instrument. This may be during the first cut phase or the second cut phase or the third cut phase. If the surgeon subsequently commands the cutting operation to resume, for example by activating an input on the surgeon input device 501, then the control system 503 may control the electrosurgical generator to restart the CUT operation by restarting the method at step 701. In other words, the CUT waveform of FIG. 8 will restart at time 0 at 0V. Alternatively, the control system 503 may control the electrosurgical generator to resume outputting the CUT waveform of the same cut phase that it was outputting prior to the surgeon commanding the cutting operation to stop.

As described above, FIG. 8 is an example in which the trigger condition of the first phase is the voltage of the CUT waveform reaching or exceeding a threshold voltage, and the further trigger condition of the second phase is both the current of the CUT waveform reaching or dropping below a current threshold and the phase angle between the current and voltage of the CUT waveform reaching or exceeding a phase angle threshold. However, in general the trigger condition of any phase of the CUT waveform may be any one or combination of the following:
1. A voltage threshold being reached;
2. A voltage threshold being exceeded;
3. A voltage threshold being dropped below;
4. A rate of change of the voltage reaching, exceeding or dropping below a threshold;
5. A current threshold being reached;
6. A current threshold being exceeded;
7. A current threshold being dropped below;
8. A rate of change of the current reaching, exceeding or dropping below a threshold;
9. A tissue impedance threshold being reached;
10. A tissue impedance threshold being exceeded;
11. A tissue impedance threshold being dropped below;
12. A rate of change of the tissue impedance reaching, exceeding or dropping below a threshold;
13. A phase angle threshold being reached;
14. A phase angle threshold being exceeded;
15. A phase angle threshold being dropped below;
16. A rate of change of the phase angle reaching, exceeding or dropping below a threshold;
17. A predetermined time period having elapsed;
18. A grip force threshold being reached;
19. A grip force threshold being exceeded;
20. A grip force threshold being dropped below; and
21. A rate of change of the grip force reaching, exceeding or dropping below a threshold.

The method described herein with reference to FIG. 7 describes two trigger conditions being monitored and responded to, one during the first cut phase and the other during the second cut phase. However, both of these trigger conditions are not necessarily required. For example, only the trigger condition during the first phase may be monitored and responded to. In this case, the surgeon may command the cutting operation to end by manipulating an input on the surgeon input device. This is instead of the control device determining to end the cutting operation a predetermined time period after a further trigger condition is met in the second cut phase. As another example, only the trigger condition during the second phase may be monitored and responded to. In this case, the surgeon may command the cutting operation to switch from the first cut phase to the second cut phase by manipulating an input on the surgeon input device. This is instead of the control device determining to switch from the first cut phase to the second cut phase a predetermined time period after a trigger condition is met in the first cut phase. As another example, rather than having phase A and phase B as shown in FIG. 8, the CUT waveform may ramp up from zero volts to a constant RMS voltage V_{EA}, and maintain that constant voltage. Upon a trigger condition being met, a timer is started, and a predetermined time period after that timer is started, the CUT waveform is dropped to zero volts. In this example, there is only one trigger condition being met and responded to. There is also only one constant RMS voltage applied to the electrosurgical instrument. In other words, there is no step down from the constant RMS voltage V_{EA} to the constant RMS voltage V_{EB}.

In the example of FIG. 8, during the period of the first cut phase whilst the timer is activated, the electrosurgical generator generates a constant RMS voltage electrosurgical signal V_{EA}. The constant RMS voltage during this timer period is the same as the trigger voltage V_{EA}. Alternatively, the constant RMS voltage during this timer period may be lower than the trigger voltage V_{EA}. Alternatively, the constant RMS voltage during this timer period may be higher than the trigger voltage V_{EA}. In the case that the trigger condition is a non-voltage condition, such as an impedance condition, then the voltage during the timer period may be a maximum voltage of the first cut phase which is higher than the voltage reached prior to the trigger condition being met. One or more electrosurgical parameter may be modified whilst the timer is activated. For example, during the period of the first cut phase whilst the timer is activated, the voltage may be ramped down from the trigger voltage V_{EA} to a lower voltage (for example OV).

As discussed in the background section, high voltage CUT waveforms used by bipolar electrosurgical instruments can cause sparking to occur. This can cause tissue surrounding the bipolar electrosurgical instruments to burn. A continuous application of high voltage may also change the surrounding media, thereby increasing the likelihood of sparks forming. The methods described herein describe implementing two cut phases, a first with a higher voltage, followed by a second with a lower voltage. The cutting operation is most effectively carried out during the first phase with the higher voltage. Once this high voltage is reached, a timer is used to keep the energy at that voltage for a predetermined time period. Use of the second phase with the lower voltage then enables the cutting operation to be completed without sparks forming. The lower voltage of the second phase may also aid coagulation of the surrounding tissue.

As described above, the bipolar electrosurgical instrument may be overmoulded with insulation material where the electrosurgical elements terminate at the electrosurgical end effector elements. The video feed viewed by the surgeon on the surgeon's display 502 from the endoscopic camera may provide little or no visibility of the tissue grasped by the end effector elements during an electrosurgical procedure due to the overmoulded areas blocking the view. Additionally, the cut tissue can remain attached to the end effector elements even after it has been successfully cut through. It can thus be difficult for the surgeon to tell when the tissue has been successfully cut through. The further trigger condition of the second cut phase described herein and subsequent ending of the cutting operation thus aids the surgeon in ensuring that the CUT waveform is applied for the correct time, i.e. long enough to cut the tissue but not so long as to damage surrounding tissue.

The phase angle and current thresholds described herein are indicative of how much electrosurgical energy is going through the tissue to cut it—i.e. active power—and how much of the electrosurgical energy is dissipated elsewhere, for example capacitively charging the instrument and surrounding media—i.e. reactive power. When the tissue is cut through, most of the measured electrosurgical power is capacitive and the current lags the voltage by almost 90° in phase angle. Thus, the phase angle between the measured voltage and current reaching or exceeding a phase angle threshold is indicative of the tissue having been substantially cut through. The measured current is indicative of the tissue impedance. As it is cut by energy, the tissue dries out, causing the current to drop. Thus, the measured current dropping below a current threshold is indicative of the tissue having been substantially cut through. Thus, one or both of the phase angle and current thresholds are usefully used to initiate a timer. Following the delay measured by the timer, the CUT waveform is dropped to zero volts, thereby ending the cutting operation. Thus, this ensures that the energy is applied to the tissue long enough to cut it through fully but not so long as to damage the surrounding tissue.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system configured to control the application of energy from an electrosurgical generator to a robotic electrosurgical instrument during a cut mode in which the electrosurgical instrument is used to cut tissue, the control system being configured to:
control the electrosurgical generator to apply energy to the electrosurgical instrument during a first cut phase;
detect a trigger condition has been met during the first cut phase, wherein the trigger condition comprises the voltage applied by the electrosurgical generator to the electrosurgical instrument reaching a voltage threshold;
upon detecting that the trigger condition has been met, start a timer; and
upon a predetermined time period elapsing after the timer has started, control the electrosurgical generator to switch from the first cut phase to a second cut phase, the second cut phase differing from the first cut phase by one or more energy parameters.

2. The control system as claimed in claim 1, wherein the trigger condition comprises both:
the current applied by the electrosurgical generator to the electrosurgical instrument reaching or dropping below a current threshold; and
the phase angle between the current and voltage applied by the electrosurgical generator to the electrosurgical instrument reaching or exceeding a phase angle threshold.

3. The control system as claimed in claim 1, wherein the trigger condition comprises the tissue impedance reaching or exceeding a tissue impedance threshold.

4. The control system as claimed in claim 1, wherein the first cut phase differs from the second cut phase by one or more of the following energy parameters: voltage limit of the voltage applied by the electrosurgical generator, power limit of the power applied by the electrosurgical generator, and current limit of the current applied by the electrosurgical generator.

5. The control system as claimed in claim 1, wherein the first cut phase is the initial application of energy from zero voltage to a first voltage, and wherein the second cut phase is the application of energy at a second voltage.

6. The control system as claimed in claim 5, wherein the first voltage is a peak voltage of the cut mode, and the second voltage is lower than the first voltage.

7. The control system as claimed in claim 5, wherein the second voltage is a peak voltage of the cut mode, and the first voltage is lower than the second voltage.

8. The control system as claimed in claim 5, wherein:
the trigger condition comprises the voltage applied by the electrosurgical generator to the electrosurgical instrument reaching or exceeding a voltage threshold; and
once the voltage threshold is reached or exceeded, the control system comprises applying the first voltage until the predetermined time period has elapsed.

9. The control system as claimed in claim 5, being further configured to:
detect a further trigger condition has been met during the second cut phase;
upon detecting that the further trigger condition has been met, start a further timer; and
upon a further predetermined time period elapsing after the further timer has started, control the electrosurgical generator to switch from the second cut phase to a third cut phase, the third cut phase differing from the second cut phase by one or more energy parameters.

10. The control system as claimed in claim 9, wherein the third cut phase is the application of zero voltage.

11. The control system as claimed in claim 9, wherein the further trigger condition comprises both:
the current applied by the electrosurgical generator to the electrosurgical instrument reaching or dropping below a current threshold; and
the phase angle between the current and voltage applied by the electrosurgical generator to the electrosurgical instrument reaching or exceeding a phase angle threshold.

12. The control system as claimed in claim 1, wherein the first cut phase is the application of energy at a first voltage, and wherein the second cut phase is the application of zero voltage.

13. The control system as claimed in claim 12, wherein the first voltage is a peak voltage of the cut mode.

14. The control system as claimed in claim 12, wherein the first voltage is lower than a peak voltage of the cut mode.

15. The control system as claimed in claim 12, wherein:
the trigger condition comprises both:
the current applied by the electrosurgical generator to the electrosurgical instrument reaching or dropping below a current threshold; and
the phase angle between the current and voltage applied by the electrosurgical generator to the electrosurgical instrument reaching or exceeding a phase angle threshold; and
once the trigger condition is met, the control system is configured to apply the first voltage until the predetermined time period has elapsed.

16. The control system as claimed in claim 12, further being configured to:
control the electrosurgical generator to apply energy to the electrosurgical instrument during a third cut phase;
detect a further trigger condition has been met during the third cut phase;
upon detecting that the further trigger condition has been met, start a further timer; and
upon a further predetermined time period elapsing after the further timer has started, control the electrosurgical generator to switch from the third cut phase to the first cut phase, the third cut phase differing from the first cut phase by one or more energy parameters.

17. The control system as claimed in claim 16, wherein the third cut phase is the initial application of energy from zero voltage to a third voltage.

18. The control system as claimed in claim 17, wherein the third voltage is a peak voltage of the cut mode.

19. The control system as claimed in claim 16, wherein the further trigger condition comprises the voltage applied by the electrosurgical generator to the electrosurgical instrument reaching or exceeding a voltage threshold.

20. (canceled)

21. (canceled)

22. (canceled)

23. (canceled)

24. The control system as claimed in claim 1, being further configured to output a cut complete indication, that cut complete indication being different to the video feed from a camera recording the tissue being cut.

25. (canceled)

26. (canceled)
